(19) United States
(12) Patent Application Publication
(10) Pub. No.: US 2024/0310535 A1
(43) Pub. Date: Sep. 19, 2024

(54) SCINTILLATOR PLATE, RADIATION DETECTOR, AND MANUFACTURING METHOD OF SCINTILLATOR PLATE

(71) Applicant: CANON KABUSHIKI KAISHA, Tokyo (JP)

(72) Inventor: TOMOYUKI OIKE, Kanagawa (JP)

(21) Appl. No.: 18/670,192

(22) Filed: May 21, 2024

Related U.S. Application Data
(63) Continuation of application No. PCT/JP2022/041932, filed on Nov. 10, 2022.

(30) Foreign Application Priority Data
Nov. 24, 2021 (JP) 2021-190617

(51) Int. Cl. G01T 1/20; G01T 1/202

(52) U.S. Cl. CPC G01T 1/2018; G01T 1/202

(57) ABSTRACT

A scintillator plate includes a columnar crystal formed on a substrate and configured to emit light based on irradiating radiation, the columnar crystal having a predetermined column diameter, and a protective film formed to cover the columnar crystal and configured to protect the columnar crystal from water vapor, the protective film having a predetermined thickness. An arithmetic average roughness of the protective film is less than or equal to the column diameter, and a thickness of the protective film is less than or equal to 4 times the column diameter.

### CROSS-REFERENCE TO RELATED APPLICATIONS

This application is a Continuation of International Patent Application No. PCT/JP2022/041932, filed Nov. 10, 2022, which claims the benefit of Japanese Patent Application No. 2021-190617, filed Nov. 24, 2021, both of which are hereby incorporated by reference herein in their entirety.

### BACKGROUND OF THE INVENTION

### Field of the Invention

The present invention relates to a scintillator plate, a radiation detector, and a manufacturing method of a scintillator plate.

### Background Art

Flat panel detectors (FPDs) used for X-ray imaging in medical settings receive X-rays passed through an object with a phosphor (scintillator plate) that is a radiation detection material formed on a substrate, and detect light emitted from the phosphor with light receiving elements. To efficiently transmit the emitted light to the light receiving elements, a group of columnar or needle-shaped crystals of halogenated alkali metal such as cesium iodide, formed by vapor deposition, has been used for the crystal part of the phosphor.

In the deposition process of cesium iodide columnar crystals, tiny crystal nuclei are typically formed on the substrate at the initial stage of deposition. By selecting the substrate temperature, pressure, and deposition rate, the crystal nuclei are preferentially grown in the <100>orientation to form columnar crystals. At the later stage of deposition, the columnar crystals increase in diameter. The group of columnar crystals has gaps between the individual columnar crystals. Due to the ratio between the refractive index of cesium iodide (approximately 1.8) and the refractive index of air (1.0), the light is repeatedly totally reflected inside the cesium iodide columnar crystals of high refractive index and effectively guided to the light receiving elements.

The group of columnar crystals has a high aspect ratio, with an extremely large specific surface area compared to ordinary flat films. A group of columnar crystals made of deliquescent halogenated alkali metal in particular deliquesces easily when exposed to water vapor in the air, and the columnar crystals get fused together or the sidewalls lose flatness. The deliquesced halogenated alkali metal can lower the spatial resolution of the radiation detector since the emitted light propagates between columnar crystals before reaching the light receiving elements or scatters at the interface.

Patent literature 1 discusses a technique for forming a first protective layer of metal alkoxide on a phosphor including a group of columnar crystals so that the first protective layer is chemically bonded with the interface with the columnar crystals, and further sealing the first protective layer with a second protective layer. The two protective layers prevent the phosphor from contacting water vapor, thereby preventing deliquescence. This enables the prevention of both a drop in the moisture-proofness of the phosphor and a drop in the spatial resolution in a compatible manner.

### CITATION LIST

### Patent Literature

PTL 1: Japanese Patent Application Laid-Open No. 2020-041820

As discussed in Patent literature 1, the first protective layer formed of metal alkoxide by chemical bonding has a thickness as thin as a monomolecular layer and still contains unreacted materials. This makes it difficult to provide a permanent moisture-proof function. To achieve desired moisture-proofness, the uneven surface of the columnar crystals is therefore desirably securely covered with the poly-para-xylylene second protective layer that is thicker than the first protective layer. As a result, the image quality of the radiographic image can drop due to the increased optical distance and increased optical interfaces, and insufficient adhesion between the first and second protective layers.

### SUMMARY OF THE INVENTION

The present invention is directed to providing a scintillator plate that can achieve high resolution and moisture-proofness in a compatible manner.

According to an aspect of the present invention, a scintillator plate includes a columnar crystal formed on a substrate and configured to emit light based on irradiating radiation, the columnar crystal having a predetermined column diameter, and a protective film formed to cover the columnar crystal and configured to protect the columnar crystal from water vapor, the protective film having a predetermined thickness, wherein an arithmetic average roughness of the protective film is less than or equal to the predetermined column diameter, and the predetermined thickness is less than or equal to 4 times the predetermined column diameter.

Further features of the present invention will become apparent from the following description of exemplary embodiments with reference to the attached drawings.

### BRIEF DESCRIPTION OF THE DRAWINGS

FIG. 1A is a sectional view illustrating how a group of column crystals according to an exemplary embodiment of the present invention is covered with a protective film.

FIG. 1B is a sectional view illustrating how the group of column crystals according to the exemplary embodiment of the present invention is covered with the protective film.

FIG. 1C is a sectional view illustrating how the group of column crystals according to the exemplary embodiment of the present invention is covered with the protective film.

FIG. 2 is a sectional scanning electron microscope (SEM) image of a surface side of the group of columnar crystals according to the exemplary embodiment of the present invention.

FIG. 3A is a sectional view schematically illustrating the ends of the columnar crystals according to the exemplary embodiment of the present invention.

FIG. 3B is a sectional view schematically illustrating the ends of the columnar crystals according to the exemplary embodiment of the present invention.

FIG. 4A is a sectional view illustrating a configuration of a radiation detector according to the exemplary embodiment of the present invention.

FIG. 4B is a sectional view illustrating a configuration of the radiation detector according to the exemplary embodiment of the present invention.

FIG. 5 is a chart illustrating temporal changes in the spatial resolution of radiation detectors according to the exemplary embodiment of the present invention.

FIG. 6 is a diagram illustrating an example of the use of a radiographic apparatus according to the exemplary embodiment of the present invention.

FIG. 7 is an example of a sectional SEM image illustrating the ends of the columnar crystals according to the exemplary embodiment of the present invention being covered with a protective film.

### DESCRIPTION OF THE EMBODIMENTS

Configurations of examples and comparative examples of the present invention will hereinafter be described.

### Surface Roughness of Phosphor

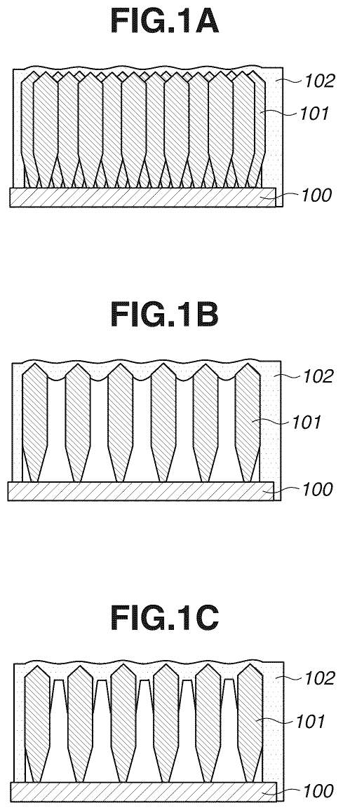

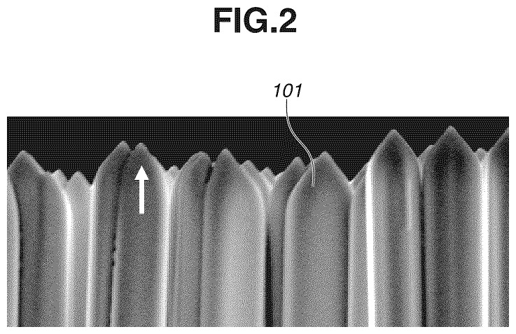

FIG. 1A illustrates a phosphor including an aggregate of columnar crystals 101 formed on a substrate. FIG. 1B illustrates a section of the aggregate. According to an exemplary embodiment of the present invention, the phosphor has an uneven surface as illustrated in FIGS. 1A and 1B. For example, the measurements of the tip angles of the columnar crystals in a sectional scanning electron microscope (SEM) image of FIG. 2 have a wide distribution, ranging from 52° to 89°. Most of the angles are within the range of 70° to 79°, with an average of 75°. The arrow in FIG. 2 indicates the growth direction of the columnar crystals 101.

In general, an average distance from a reference line within a sampling interval is referred to as an arithmetic average roughness (Ra), with the average of the surface unevenness as the reference line. Ra is determined by the following equation (Eq. 1):

$$Ra ( μ m ) = \frac{1}{L} {\int }_{0}^{L} \vert f ( x ) \vert dx , \qquad ( Eq . 1 )$$

where the x-axis is in the direction of the average line within a sampling interval L and the y-axis is in the direction of the vertical magnification, and the roughness curve is expressed as y=f(x). The smaller the value of Ra, the flatter and the smoother the surface.

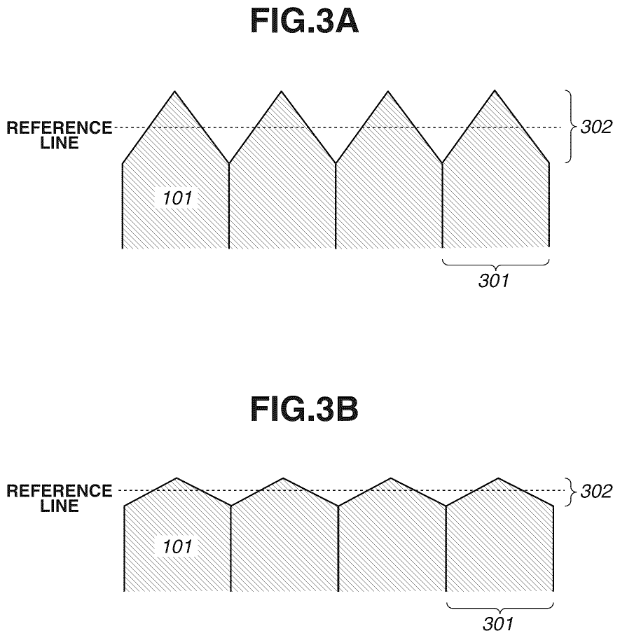

For example, if, as illustrated in FIG. 3A, columnar crystals 101 of the phosphor with a column diameter 301 of 10 μm, a projection height 302 of 7 μm, and a tip angle of 71° are compactly arranged, Ra is 1.75 μm. If, as illustrated in FIG. 3B, the surface is shaped smoother than in FIG. 3A, with a tip angle of 104° and a projection height of 3.9 μm, Ra is approximately 0.98 μm, which is a value smaller than that of the phosphor illustrated in FIG. 3A.

A protective film 102 according to the present exemplary embodiment is formed on the phosphor surface so that the value of the surface roughness of the projective film 102 is less than or equal to the column diameter 301. This reduces the surface roughness at the phosphor surface to make the surface smoother and improve the stability of the next process. The value of the surface roughness of the protective film 102 can desirably be 10% or less of the column diameter 301.

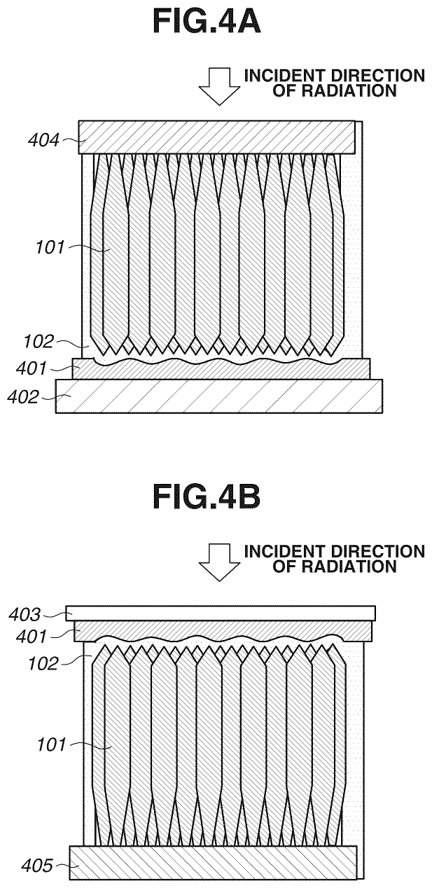

For example, in fabricating an indirect flat panel detector of FIG. 4A, the reduced surface roughness at the phosphor surface improves adhesion when the phosphor is bonded to an optical sensor 402 via an adhesive layer 401. Similarly, in fabricating a direct flat panel detector of FIG. 4B, the reduced surface roughness improves adhesion when a reflection layer 403 is formed via an adhesive layer 401.

Like the present exemplary embodiment, even if the phosphor surface is not perfectly planarized, the surface roughness of the protective film 102 can be sufficiently absorbed since the typical adhesive layer 401 has a thickness of several micrometers or more. The adhesive layer 401 may be formed of a hot melt resin or an adhesive material as appropriate.

Since the adhesive layer and constituent members in use can be reduced in thickness, the length (optical length) from the ends of the columnar crystals 101 to the surface of the optical sensor 402 can be further reduced. This also provides the effect of improving the spatial resolution of the flat panel detector.

Since the protective film 102 according to the present exemplary embodiment can reduce the surface roughness of the phosphor, the refraction angles of light at the interface between the columnar crystals 101 and the protective film 102 decrease, especially at the projections at the ends of the columnar crystals 101. This increases the components of the guided light that gently change their direction toward the growth direction of the columnar crystals 101. As a result, diffusion of the light incident on the optical sensor 402 can be prevented.

The surface roughness can be measured using a contact stylus surface roughness meter or a non-contact confocal laser microscope. The latter capable of nondestructive measurement through light irradiation is desirably used to measure soft and brittle surfaces like those of the columnar crystals 101.

### Thickness of Protective Film

If, for example, the columnar crystals 101 of the phosphor have a column diameter of 10 μm and a tip angle of 89°, the projection height is 5.09 μm, which is approximately 0.5 times the column diameter. This value serves as a lower guideline for the protective film 102 in covering the range from the tips to the sidewall portions of the columnar crystals 101. If the protective film 102 is thinner than the lower guideline, insufficient coating of the tips of the columnar crystals 101 can undesirably lower the moisture-proofness of the columnar crystals 101.

If the columnar crystals 101 have a column diameter of 10 μm and a tip angle of 52°, the projection height is 10.3 μm. This value is substantially the same as the column diameter, and servers as an upper guideline for the protective film 102 in covering the range from the tips to the sidewall portions of the columnar crystals 101. If the protective film 102 is thicker than the upper guideline and the gaps between the columnar crystals 101 are filled with the protective film, the spatial resolution drops undesirably since the emitted light can be guided or scattered between the plurality of columnar crystals 101.

In fact, there are columnar crystals 101 of various column diameters, ranging from several micrometers to several tens of micrometers. The upper limit is thus desirably less than or equal to four times the column diameter. The protective film 102 therefore desirably have a thickness value 0.5 times or more and 4 times or less of the column diameter value.

### Coverage of Protective Film

The coverage of the protective film 102 in the thickness direction of the phosphor according to the present exemplary embodiment will now be described. Suppose, for example, the columnar crystals 101 are formed on a substrate by vapor deposition. Since columnar crystals grown from fine-diameter crystal nuclei form the group of columnar crystals 101 through gradual selection and fusion, the column diameters of the columnar crystals 101 increase gradually with thickness. The distances between columnar crystals are extremely small when the columnar crystals are fine crystal nuclei. As the thickness increases, the columnar crystals become more separated from each other, and beyond a certain thickness, the distances between the columnar crystals are maintained at a relatively constant level.

If the protective film 102 according to the present exemplary embodiment is formed on the area including fine-diameter crystal nuclei at the initial stage of vapor deposition, the gaps between the columnar crystals 101 are filled with the protective film 102 and disappear. The light to be guided is thus scattered, and the spatial resolution drops. As illustrated in FIG. 1C, the coverage of the protective film 102 is therefore desirably such that the columnar crystals 101 are sufficiently separated from each other with clear gaps and the sidewalls of the columnar crystals 101 can be coated with the protective film 102 while maintaining the gaps between the columnar crystals 101. The protective film 102 desirably coats the sidewalls of the ends of the columnar crystals 101 by 50% or less of the thickness of the phosphor.

### Material of Protective Film

The material of the protective film 102 used in the present exemplary embodiment can be a liquid containing a polysilazane-based inorganic polymer composed of silicon, nitrogen, and hydrogen, such as perhydropolysilazane. The liquid material can be formed by adjusting the concentration using an organic solvent with various catalysts added as appropriate. Depending on the types of materials selected, the solution may be heated as appropriate during hydrolysis reaction or conversion reaction into silica glass. For simplicity, materials that cause the foregoing reaction at room temperature are preferably selected.

### Method for Forming Protective Film

Liquid-based techniques such as spin coating, spray coating, dip coating, flow coating, and bar coating can be selected as appropriate for the formation process. This enables easy coating of the columnar crystals 101 with a desired thickness compared to vapor phase deposition techniques, for example. The use of liquid material facilitates supplying a large amount of material to the surfaces, or in particular recessed surfaces of the group of columnar crystals 101. The surface roughness due to the surface unevenness of the columnar crystals 101 can thereby be reduced.

For example, spin coating increases the time for the applied material solution to remain at the ends of the columnar crystals 101 due to the centrifugal force generated by the rotation. This desirably facilitates the formation of the protective film 102 on the tips of the columnar crystals 101. This can also minimize the formation of the protective film 102 between the columnar crystals 101.

Spray coating can uniformize the amount of application of the material solution to a large-area substrate. Dip coating enables thick application of the material solution to both sides of the substate at a time.

The formation of the protective film 102 between the columnar crystals 101 can also be reduced by holding the plurality of columnar crystals 101 with the tips vertically downward during material application and during drying. To promote chemical reaction, the columnar crystals 101 may be warmed, heated, or mildly humidified without causing deliquescence of the columnar crystals 101 as appropriate during the supply and application of the material and after the formation of the protective film 102.

Moreover, the columnar crystals 101 may be subjected to planarization treatment. Suitable methods for the planarization treatment include uniformly pressing the surface of the phosphor with a flat plate or a roller, and removing abnormally grown crystal regions. The techniques are not limited as long as the surface roughness of the phosphor can be reduced.

Before the formation of the protective film according to the present exemplary embodiment, the deposited phosphor may be coated with metal alkoxides such as ethyl silicate to prevent degradation in characteristics during a time until the completion of the next step and during a storage period.

### Phosphor Material

The base material of the phosphor can be selected from halogenated alkali metal compounds that can form the group of columnar crystals 101, such as cesium iodide and cesium bromide. To provide the base material with a sufficient luminescence function, activators such as thallium iodide and thallium bromide can be included into the base material. The phosphor according to the present exemplary embodiment can be fabricated using a common vacuum deposition technique such as vapor deposition.

### Evaluation

A shape evaluation as to the column diameter 301 of the columnar crystals 101 of the fabricated phosphor can be suitably made using a SEM. To evaluate resolution characteristics, the modulation transfer function (MTF) can be measured for quantitative comparison. The detective quantum efficiency (DQE) can be evaluated using various light receiving elements such as charge-coupled device (CCD) and complementary metal-oxide-semiconductor (CMOS) elements, and optical detectors such as a camera. The chemical composition of the evaporated film can be evaluated by X-ray fluorescence analysis or inductively coupled plasma analysis, for example. The crystallinity can be evaluated by X-ray diffraction analysis, for example.

### Application for Radiation Detector and Radiographic Apparatus

An example of a radiation detector using a scintillator plate according to the present exemplary embodiment is an indirect radiation detector illustrated in FIG. 4A, where the phosphor is formed on a substrate 404 accompanied with a reflection layer, and combined with an optical sensor 402 for converting light into charges via an adhesive layer 401. Another example is a direct radiation detector illustrated in FIG. 4B, where the phosphor is formed on a substrate 405 accompanied with an optical sensor, and combined with a reflection layer 403 via an adhesive layer 401.

The foregoing radiation detector can be applied to a radiographic apparatus for capturing an image based on radiation. While X-rays are typically used for the radiation, alpha rays or beta rays may be used instead.

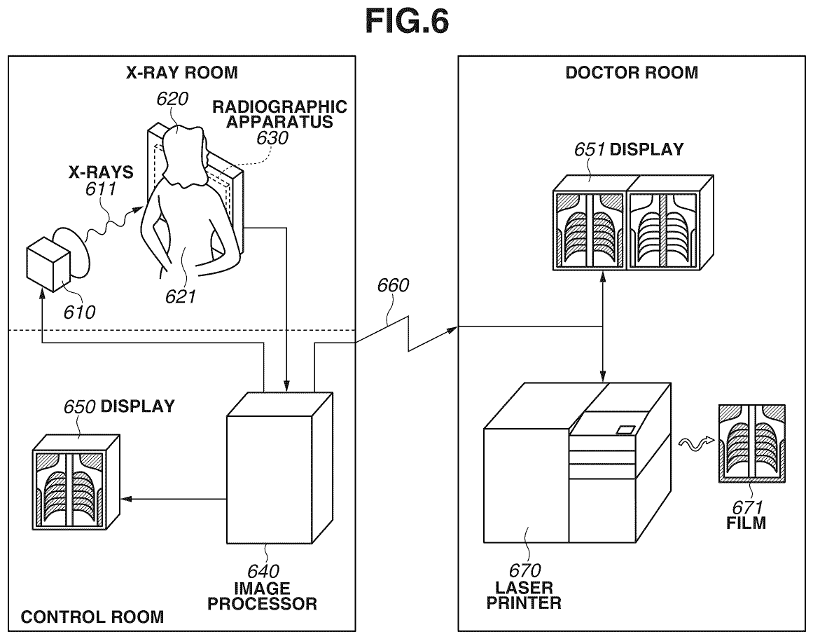

FIG. 6 illustrates an example of the use of the radiographic apparatus. Radiation 611 generated by a radiation source 610 is transmitted through a chest 621 of a patient 620 and incident on a radiographic apparatus 630. The radiation 611 incident on the radiographic apparatus 630 contains information about the internal anatomy of the patient 620. The radiographic apparatus 630 obtains electrical information based on the radiation 611. This electrical information is converted into a digital signal and then subjected to predetermined image processing by an image processor 640, for example.

A user such as a doctor can observe a radiographic image based on the electrical information on a display 650 in a control room, for example. The user can transfer the radiographic image or its data to a remote location using predetermined communication means 660. This radiographic image can be observed on a display 651 in a doctor room that is another location. The user can also record the radiographic image or its data on a predetermined recording medium. For example, the radiographic image or data can be recorded on a film 671 by a film processor 670.

Comparative examples and practical examples of the present exemplary embodiment will now be described.

### First Comparative Example

In this comparative example, a phosphor having a columnar crystal structure was formed with cesium iodide as a base material and thallium iodide as an activator, using a vacuum deposition apparatus. A protective film of ethyl silicate was then formed.

A material source filled with cesium iodide as an evaporation base material, a material source filled with thallium iodide as an evaporation activator material, and a substrate were initially placed in the vacuum deposition apparatus. The substrate was a glass substrate on which a 100-nm-thick aluminum reflection layer and a 50-nm-thick silicon dioxide layer were stacked.

The vacuum deposition apparatus was evacuated to 0.01 Pa or less inside, and a current was gradually flowed through each material source for heating. When the temperatures of the material sources reached set temperatures, the substrate was rotated and shutters between the substrate and the material sources were opened to start deposition. The substrate temperature was gradually increased from 80° C. to 160° C. The state of deposition was monitored, and when a desired thickness was reached, the shutters were closed to end the deposition. After the substrate and the material sources were cooled to room temperature, the deposition film was quickly exposed to ethyl silicate and coated with the protective film by vapor phase deposition.

The deposition film was observed under a SEM, and the formation of a group of columnar crystals as illustrated in FIG. 2A was confirmed. The film surface of the deposition film was brought into close contact with a CMOS optical detector via a fiber optic plate (FOP) and the substrate was irradiated with X-rays conforming to the international standard beam quality RQA5 to obtain an image. MTF(2) at a spatial frequency of 2 Lp/mm, which is an index for the resolution of a radiation detection material, was determined by the edge method using a tungsten knife edge.

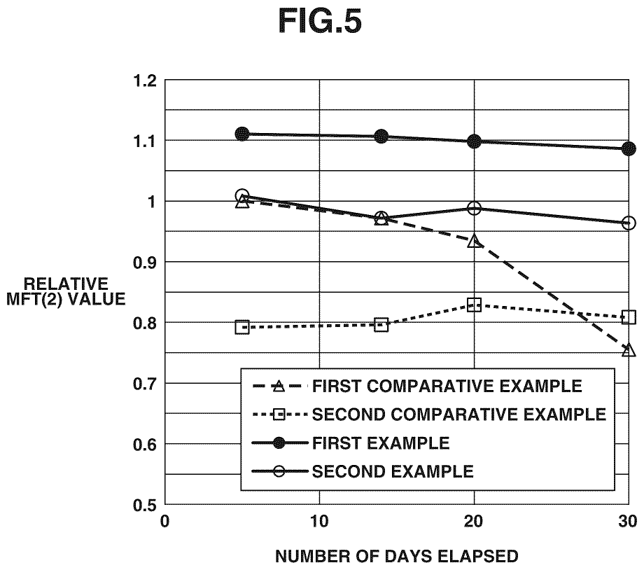

The article was stored in an environment of 25° C. and 50%-humidity, and measured for MTF daily. The graph of FIG. 5 illustrates the obtained temporal change curve of the first comparative example, with the value of MTF(2) after a lapse of five days as 1. The relative MFT(2) value decreased gradually and fell to 0.76 after 30 days, showing a drop in the spatial resolution.

### Second Comparative Example

In this comparative example, a phosphor having a columnar crystal structure was formed with cesium iodide as a base material and thallium iodide as an activator, using a vacuum deposition apparatus. A protective film of ethyl silicate was then formed, and a protective film of poly-para-xylylene was further formed thereon.

The deposition film and the ethyl silicate protective film were formed on a substrate accompanied with a reflection film in a manner similar to that of the first comparative example. The substrate was taken out and then loaded into a poly-para-xylylene deposition apparatus. After vacuum evacuation, radical para-xylylene obtained by thermally activating material di-para-xylylene was introduced into the deposition chamber, whereby a 15-μm-thick poly-para-xylylene protective film was formed on the surface.

The change of the MTF(2) over time was measured in a manner similar to that of the first comparative example. The graph of FIG. 5 illustrates the obtained temporal change curve of the second comparative example with the value of MTF(2) of the first comparative example after a lapse of five days as 1. The relative MFT(2) value after five days was as low as 0.79 due to the thick poly-para-xylylene protective film. However, the resolution decreased little even after 30 days, which shows moisture-proofness.

### First Example

This first example is an example of a manufacturing method of a scintillator plate according to the present exemplary embodiment. A phosphor including an aggregate of columnar crystals 101 was formed with cesium iodide as a base material and thallium iodide as an activator, using a vacuum deposition apparatus. A protective film 102 of perhydropolysilazane was then formed.

As a preparation step, a phosphor deposition film was initially formed on a substrate accompanied with a reflection film. The deposition film was formed in a manner similar to that of the first comparative example.

Next, as a protective film formation step, the substrate was taken out and quickly loaded into a spin coater. The protective film 102 was formed by spin coating using a material solution containing a small amount of perhydropolysilazane in a dibutyl ether solvent. The article was dried for 24 hours in an environment of 25° C. and 50%-humidity.

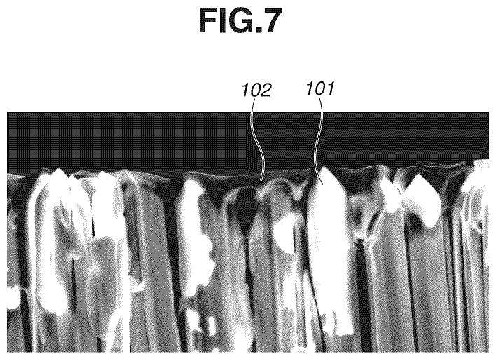

FIG. 7 illustrates an example of the deposition film observed under a SEM, where the surface of the phosphor including an aggregate of columnar crystals 101 was covered with the integral protective film 102. The change of MTF(2) over time was measured in a manner similar to that of the first comparative example. The graph of FIG. 5 illustrates the obtained temporal change curve of the first example with the value of MTF(2) of the first comparative example after a lapse of five days as 1. This shows higher resolution than in the first comparative example with little degradation in the resolution even after 30 days, and the high resolution and moisture-proofness are found to be achievable in a compatible manner.

### Second Example

This second example is an example of the manufacturing method of a scintillator plate according to the present exemplary embodiment. A phosphor including an aggregate of columnar crystals 101 was formed with cesium iodide as a base material and thallium iodide as an activator, using a vacuum deposition apparatus. A protective film of ethyl silicate was formed thereon. Abnormally grown crystal regions were then planarized, and a protective film 102 of perhydropolysilazane was further formed.

As a preparation step, a phosphor deposition film was initially formed on a substrate accompanied with a reflection film and a protective film of ethyl silicate was further formed in a manner similar to that of the first comparative example.

As a planarization step, the substrate was taken out to planarize the abnormally grown crystal regions of the phosphor. The substrate was sandwiched between 2-mm-thick glass plates and placed in a vacuum pressurization apparatus, and pressurized to 0.1 MPa.

Next, as a protective film formation step, the protective film 102 was formed by spray coating using a material solution containing a small amount of perhydropolysilazane in a dibutyl ether solvent. The article was dried for three hours at 50° C.

The change of MTF(2) over time was measured in a manner similar to that of the first comparative example. The graph of FIG. 5 illustrates the obtained temporal change curve of the second example with the value of MTF(2) of the first comparative example after a lapse of five days as 1. This shows that the resolution was maintained with little degradation even after 30 days, and the high resolution and moisture-proofness are achievable in a compatible manner.

While several suitable examples have been described above, the present invention is not limited thereto, and alterations may be made without departing from the gist of the present invention. The terms described in this specification are used solely for the purpose of describing the present invention, and it will be understood that the present invention is not limited to the strict meanings of the terms and can include equivalents thereof.

The present invention is not limited to the above embodiments and various changes and modifications can be made within the spirit and scope of the present invention. Therefore, to apprise the public of the scope of the present invention, the following claims are made.

The foregoing features enable a scintillator plate to achieve both high resolution and moisture-proofness in a compatible manner.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A scintillator plate comprising:
a columnar crystal formed on a substrate and configured to emit light based on irradiating radiation, the columnar crystal having a predetermined column diameter; and
a protective film formed to cover the columnar crystal and configured to protect the columnar crystal from water vapor,
wherein an arithmetic average roughness of the protective film is less than or equal to the predetermined column diameter, and a thickness of the protective film is less than or equal to 4 times the predetermined column diameter.

2. The scintillator plate according to claim 1, wherein the arithmetic average roughness of the protective film is less than or equal to 10% of the predetermined column diameter, and the thickness of the protective film is greater than or equal to 0.5 times and less than or equal to 4 times the predetermined column diameter.

3. The scintillator plate according to claim 1, wherein the protective film coats a sidewall of the columnar crystal by 50% or less of a height of the columnar crystal from a tip of the columnar crystal.

4. The scintillator plate according to claim 1, wherein the protective film is made of silica.

5. The scintillator plate according to claim 1, wherein the columnar crystal is made of a halogenated alkali metal compound.

6. A radiation detector comprising:
the scintillator plate according to claim 1; and
an optical sensor configured to convert light from the scintillator plate into an electrical charge.

7. A manufacturing method of a scintillator plate, comprising:
preparing a phosphor including an aggregate of columnar crystals formed on a substrate; and
forming a protective film to cover the phosphor by applying and drying a material of the protective film,
wherein surface roughness of the columnar crystals is reduced by planarization treatment before the forming.

8. The manufacturing method according to claim 7, wherein the protective film is formed by spin coating.

9. The manufacturing method according to claim 7, wherein the protective film is formed by spray coating.

10. The manufacturing method according to claim 7, wherein the material of the protective film contains polysilazane.
